(19) United States
(12) Patent Application Publication
(10) Pub. No.: US 2018/0273452 A1
(43) Pub. Date: Sep. 27, 2018

(54) PROCESS FOR THE PREPARATION OF GLYCOLS

(71) Applicant: SHELL OIL COMPANY, HOUSTON, TX (US)

(72) Inventors: Johannes Leo Marie VAN DER BIJL, Rijswijk (NL); Evert VAN DER HEIDE, Amsterdam (NL); Pieter HUIZENGA, Amsterdam (NL); Munro MACKAY, Amsterdam (NL)

(21) Appl. No.: 15/763,466

(22) PCT Filed: Sep. 27, 2016
(86) PCT No.: PCT/EP2016/073001

§ 371 Date: Mar. 27, 2018

Related U.S. Application Data
(60) Provisional application No. 62/234,108, filed on Sep. 29, 2015.

(51) Int. Cl. C07C 29/132; C07C 29/60; B01J 23/652; B01J 37/18

(52) U.S. Cl. CPC C07C 29/132; B01J 37/18; B01J 23/6527; C07C 29/60

(57) ABSTRACT

The invention provides a process for the production of glycols comprising the step of adding to a reactor vessel a saccharide-containing feedstock, a solvent, hydrogen, a retro-aldol catalyst composition and a catalyst precursor and maintaining the reactor vessel at a temperature and a pressure, wherein the catalyst precursor comprises one or more cations selected from groups 8, 9, 10 and 11 of the periodic table, and wherein the catalyst precursor is reduced in the presence of hydrogen in the reactor vessel into an unsupported hydrogenation catalyst.

### FIELD OF THE INVENTION

The present invention relates to a process for the preparation of glycols from saccharide-containing feedstocks under conditions which convert a catalyst precursor into an unsupported hydrogenation catalyst for the process.

### BACKGROUND OF THE INVENTION

Glycols such as mono-ethylene glycol (MEG) and mono-propylene glycol (MPG) are valuable materials with a multitude of commercial applications, e.g. as heat transfer media, antifreeze, and precursors to polymers, such as PET. Ethylene and propylene glycols are typically made on an industrial scale by hydrolysis of the corresponding alkylene oxides, which are the oxidation products of ethylene and propylene, produced from fossil fuels.

In recent years, increased efforts have focussed on producing chemicals, including glycols, from non-petrochemical renewable feedstocks, such as sugar-based materials. The conversion of sugars to glycols can be seen as an efficient use of the starting materials with the oxygen atoms remaining intact in the desired product.

Current methods for the conversion of saccharides to glycols revolve around a two-step process of hydrogenolysis and hydrogenation, as described in Angew, Chem. Int. Ed. 2008, 47, 8510-8513.

Such two-step reaction requires at least two catalytic components. Patent application WO2015028398 describes a continuous process for the conversion of a saccharide-containing feedstock into glycols, in which substantially full conversion of the starting material and/or intermediates is achieved and in which the formation of by-products is reduced. In this process the saccharide-containing feedstock is contacted in a reactor vessel with a catalyst composition comprising at least two active catalytic components comprising, as a first active catalyst component with hydrogenation capabilities, one or more materials selected from transition metals from groups 8, 9 or 10 or compounds thereof, and, as a second active catalyst component with retro-aldol catalytic capabilities, one or more materials selected from tungsten, molybdenum and compounds and complexes thereof. Retro-aldol catalytic capabilities referred to herein means the ability of the second active catalyst component to break carbon-carbon bonds of sugars such as glucose to form retro-aldol fragments comprising molecules with carbonyl and hydroxyl groups. Glucose, which is an aldol product, for example, when broken into simple retro-aldol fragments yields glycolaldehyde.

It is well known in the art of chemicals manufacturing that catalysts may be described as homogeneous or heterogeneous, the former being those catalysts which exist and operate in the same phase as the reactants, while the latter are those that do not.

Typically, heterogeneous catalysts may be categorised into two broad groups. One group comprise the supported catalytic compositions where the catalytically active component is attached to a solid support, such as silica, alumina, zirconia, activated carbon or zeolites. Typically these are either mixed with the reactants of the process they catalyse, or they may be fixed or restrained within a reaction vessel and the reactants passed through it, or over it. The other group comprise catalytic compositions where the catalytically active component is unsupported, i.e. it is not attached, to a solid support, an example of this group is the Raney-metal group of catalysts. An example of a Raney-metal catalyst is Raney-nickel, which is a fine-grained solid, composed mostly of nickel derived from a nickel-aluminium alloy. The advantage of heterogeneous catalysts is that they can be retained in the reactor vessel during the process of extracting the unreacted reactants and the products from the reactor vessel, giving the operator the capability of using the same batch of catalysts many times over. However, the disadvantage of heterogeneous catalysts is that over time their activity declines, for reasons such as the loss, or leaching, of the catalytically active component from its support, or because the access of the reactants to the catalytically active component is hindered due to the irreversible deposition of insoluble debris on the catalyst's support. As their activity declines, catalysts need to be replaced, and for heterogeneous catalysts this inevitably requires the process that they catalyse to be stopped, and the reactor vessel to be opened up, to replace the deactivated catalyst with a fresh batch. Such down-time is costly to the operators of the process, as during such time no products can be produced, and such a labour-intensive operations have cost implications.

A further complication of using heterogeneous catalysts is that the process of preparing the catalyst, and in particular the process of immobilising catalytically active components onto a solid support in a way that gives maximum catalytic activity can be difficult and time consuming.

Homogeneous catalysts are typically unsupported and operate in the same phase as the reactants of the reaction they catalyse. Therefore their preparation does not require any step(s) for immobilising the catalytically active components onto a solid support, and their addition to, and mixing with, the reactants of the reaction they catalyse is much easier. However, separation of the catalyst from the reactants becomes more difficult, and in some cases not possible. This means that, in general, homogeneous catalysts either require to be replenished more often than heterogeneous catalysts, and/or additional steps and hardware are required in the process to remove the catalyst from the reactants and reaction products, with an obvious impact on the overall economy of the processes that they catalyse.

Regarding the two-step continuous process of making glycols from saccharide-containing feedstock, as described in WO2015028398, the activities and robustness of the at least two catalytic components, each of which is typically a heterogeneous catalyst, can vary with respect to each other, and therefore if the activity of any one of them declines sooner than the activity of the other, the process of glycol production will not go to completion as efficiently as it was at the commencement of the process, forcing the operators to stop the process to recharge one or both of the catalysts. Alternatively, breakdown components of one of the two catalytic components may adversely affect the other's activity. Again in such a case, the operators of the process are forced to stop the process to recharge one or both of the catalysts. A particular problem faced in this regard is the effect of insoluble tungsten and molybdenum compounds and complexes formed from the degradation of the catalyst component with retro-aldol catalytic capabilities. Such insoluble matter attach to and clog up the surface of the catalyst component with hydrogenation capability, especially if such catalyst component comprises porous solid support and/or is unsupported, but nevertheless has a porous surface topology.

It would, therefore be, advantageous to be able to prepare an unsupported hydrogenation catalyst which is suitable for the hydrogenation of retro-aldol fragments in the process for the preparation of glycols from saccharide-containing feedstock: (i) with minimal labour, including without the time consuming and tricky step of immobilisation of the catalytically active components on a solid support, (ii) which functions with the advantages of both a homogeneous-type and a heterogeneous-type catalysts, but without their respective disadvantages, and (iii) which is unaffected by insoluble chemical species originating from the degradation of the catalyst component with retro-aldol catalytic capabilities, so that the two-step process of the conversion of saccharide-containing feedstock to glycols can be carried out in one reaction vessel, thus simplifying the process.

### SUMMARY OF THE INVENTION

The present invention concerns a process for the production of glycols comprising the step of adding to a reactor vessel a saccharide-containing feedstock, a solvent, hydrogen, a retro-aldol catalyst composition and a catalyst precursor and maintaining the reactor vessel at a temperature and a pressure, wherein the catalyst precursor comprises one or more cations selected from groups 8, 9, 10 and 11 of the periodic table, and wherein the catalyst precursor is reduced in the presence of hydrogen in the reactor vessel into an unsupported hydrogenation catalyst.

The inventors of the present processes have surprisingly found that an unsupported hydrogenation catalyst for the production of glycols from a saccharide-containing feedstock can be formed ‘in situ’ by supplying a catalyst precursor into a reactor vessel containing a mixture comprising hydrogen, either at the start of glycol production from the saccharide-containing feedstock, or during it. Therefore, other than choosing the desired catalyst precursor(s) and supplying it to the reactor vessel that contains a mixture comprising hydrogen, no preparation steps are required, making the process quick and cheap, and overcomes the challenges of conventional catalyst manufacture.

Further, inventors of the present processes have surprisingly found that although the catalyst precursor can be dissolved in a solvent and such solution is not retained by filtering through a 0.45 μm pore size filter, once converted into the unsupported hydrogenation catalyst, it comprises metal particles that are retained by filtering through a 0.45 μm pore size filter. Therefore overall, it behaves as if it is both as a homogeneous catalyst and a heterogeneous catalyst. For example, the supply of the catalyst precursor into the reactor vessel is in the same phase as the saccharide-containing feedstock, as if it is a homogeneous catalyst. This overcomes the cumbersome steps of charging the reactor vessel with the heterogeneous hydrogenation catalyst. However, the unsupported hydrogenation catalyst can be removed easily from the reactor vessel, or separated from the reaction products, by a simple filtration process, as if it is a heterogeneous catalyst, thus overcoming cumbersome solids handling which would otherwise be required. This reduces the cost and complexity of the reactor vessels suitable to carry out the glycol production process of the invention.

The inventors have also found that once the glycol production is underway, the levels of the unsupported hydrogenation catalyst inside the reactor vessel can be altered at any time by either the addition of more catalyst precursor into the reactor vessel as described above, or by the removal of the unsupported hydrogenation catalyst from the reactor vessel by filtration.

The inventors of the present processes have also surprisingly found that the unsupported hydrogenation catalyst is resistant to insoluble chemical species generated during the process for the preparation of glycols from a saccharide-containing feedstock by the degradation of the catalyst component with retro-aldol catalytic capabilities. This enables the retro-aldol and the hydrogenation steps to be carried out simultaneously in the same reactor vessel, again with the advantage of simplifying the process and therefore lowering the operational and capital costs of the process.

### DETAILED DESCRIPTION OF THE INVENTION

The present invention concerns a process for the preparation of glycols from saccharide-containing feedstocks using an unsupported hydrogenation catalyst which can be generated inside a reaction vessel where the glycol production takes places (i.e. ‘in situ’) by supplying a catalyst precursor into the reaction vessel.

The catalyst precursor is a metal salt or a metal complex. In one embodiment, the catalyst precursor comprises a cation of an element selected from chromium and groups 8, 9, 10 and 11 of the periodic table. Preferably, the cation is of an element selected from the group consisting of chromium, iron, ruthenium, cobalt, rhodium, iridium, nickel, palladium, platinum and copper. More preferably the cation is of an element selected from the group comprising nickel, cobalt and ruthenium. Most preferably, the catalyst precursor comprises a ruthenium cation. In another embodiment, the catalyst precursor comprises a mixture of cations of more than one element selected from chromium and groups 8, 9, 10 and 11 of the periodic table. Preferably, the cations are of elements selected from the group consisting of chromium, iron, ruthenium, cobalt, rhodium, iridium, nickel, palladium, platinum and copper. Suitable examples of such mixture of cations may be a combination of nickel with palladium, or a combination of palladium with platinum, or a combination of nickel with ruthenium.

The catalyst precursor is a metal salt or a metal complex. In one embodiment, the catalyst precursor comprises an anion selected from the group consisting of inorganic anions and organic anions, preferably anions of carboxylic acids. In the case of both the organic and the inorganic anions, the anion must form a salt or a metal complex with the cations listed above, which is soluble in a mixture comprising the saccharide-containing feedstock, the solvent and the glycols. Preferably, the anion is selected from oxalate, acetate, propionate, lactate, glycolate, stearate, acetylacetonate, nitrate, chloride, bromide, iodide or sulphate. More preferably, the anion is selected from acetate, acetylacetonate or nitrate. Even more preferably, the anion is selected from acetate or acetylacetonate, and most preferably, the anion is acetylacetonate. In the embodiment where the catalyst precursor comprises more than one cation, the anion of each of the metal salts or metal complexes may be any one of the anions listed above, with the proviso that each metal salt or each metal complex must be soluble in a mixture comprising the saccharide-containing feedstock, the solvent and the glycols.

The catalyst precursor is preferably supplied to the reactor vessel as a solution in a solvent. Preferably, such solvent is water and/or a solution of glycols in water and/or the product stream from the reactor vessel used for the process of producing glycols described herein.

The solution of the catalyst precursor is preferably pumped into the reactor vessel and mixed together with the reactor vessel contents.

The glycols produced by the process of the present invention are preferably 1,2-butanediol, MEG and MPG, and more preferably MEG and MPG, and most preferably MEG. The mass ratio of MEG to MPG glycols produced by the process of the present invention is preferably 5:1, more preferably 7:1 at 230° C. and 8 MPa.

The saccharide-containing feedstock for the process of the present invention comprises starch. It may also comprise one or further saccharides selected from the group consisting of monosaccharides, disaccharides, oligosaccharides and polysaccharides. An example of a suitable monosaccharide is glucose, and an example of a suitable disaccharide is sucrose. Examples of suitable oligosaccharides and polysaccharides include cellulose, hemicelluloses, glycogen, chitin and mixtures thereof.

In one embodiment, the saccharide-containing feedstock for said processes is derived from corn. Alternatively, the saccharide-containing feedstock may be derived from grains such as wheat or, barley, from rice and/or from root vegetables such as potatoes, cassava or sugar beet, or any combinations thereof. In another embodiment, a second generation biomass feed such as lignocellulosic biomass, for example corn stover, straw, sugar cane bagasse or energy crops like Miscanthus or sweet sorghum and wood chips, can be used as, or can be part of, the saccharide-containing feedstock.

A pre-treatment step may be applied to the saccharide-containing feedstock to remove particulates and other unwanted insoluble matter, or to render the carbohydrates accessible for hydrolysis and/or other intended conversions.

If required, further pre-treatment methods may be applied in order to produce the saccharide-containing feedstock suitable for use in the present invention. One or more such methods may be selected from the group including, but not limited to, sizing, drying, milling, hot water treatment, steam treatment, hydrolysis, pyrolysis, thermal treatment, chemical treatment, biological treatment, saccharification, fermentation and solids removal.

After the pre-treatment, the treated feedstock stream is suitably converted into a solution, a suspension or a slurry in a solvent.

The solvent may be water, or a C1 to C6 alcohol or polyalcohol, or mixtures thereof. Suitably C1 to C6 alcohols include methanol, ethanol, 1-propanol and isopropanol. Suitably polyalcohols include glycols, particularly products of the hydrogenation reaction, glycerol, erythritol, threitol, sorbitol, 1,2-hexanediol and mixtures thereof. More suitably, the poly alcohol may be glycerol or 1,2-hexanediol. Preferably, the solvent is water.

The concentration of the saccharide-containing feedstock as a solution in the solvent supplied to the reactor vessel is at most at 80% wt., more preferably at most at 60% wt. and more preferably at most at 45% wt. The concentration of the saccharide-containing feedstock as a solution in the solvent supplied to the reactor vessel is at least 5% wt., preferably at least 20% wt. and more preferably at least 35% wt.

The process for the preparation of glycols from a saccharide-containing feedstock requires at least two catalytic components. The first of these is a catalyst component with retro-aldol catalytic capabilities as described in patent application WO2015028398. The role of this catalyst in the glycol production process is to generate retro-aldol fragments comprising molecules with carbonyl and hydroxyl groups from the sugars in the saccharide-containing feedstock, so that the unsupported hydrogenation catalyst can convert the retro-aldol fragments to glycols.

Preferably, the active catalytic components of the catalyst component with retro-aldol catalytic capabilities comprises of one or more compound, complex or elemental material comprising tungsten, molybdenum, vanadium, niobium, chromium, titanium or zirconium. More preferably the active catalytic components of the catalyst component with retro-aldol catalytic capabilities comprises of one or more material selected from the list consisting of tungstic acid, molybdic acid, ammonium tungstate, ammonium metatungstate, ammonium paratungstate, sodium phosphotungstate, sodium metatungstate, tungstate compounds comprising at least one Group I or II element, metatungstate compounds comprising at least one Group I or II element, paratungstate compounds comprising at least one Group I or II element, phosphotungstate compounds comprising at least one Group I or II element, heteropoly compounds of tungsten, heteropoly compounds of molybdenum, tungsten oxides, molybdenum oxides, vanadium oxides, metavanadates, chromium oxides, chromium sulphate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the second active catalyst component comprises one or more compound, complex or elemental material selected from those containing tungsten or molybdenum.

In one embodiment, the active catalytic components of the catalyst component with retro-aldol catalytic capabilities is supported on a solid support, and operates as a heterogeneous catalyst. The solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

In another embodiment, the active catalytic component of the catalyst component with retro-aldol catalytic capabilities is unsupported, and operates as a homogeneous catalyst. Preferably, in this embodiment the active catalytic components of the catalyst component with retro-aldol catalytic capabilities is metatungstate, which is delivered into the reactor vessel as an aqueous solution of sodium metatungstate.

Suitable reactor vessels that can be used in the process of the preparation of glycols from a saccharide-containing feedstock include continuous stirred tank reactors (CSTR), plug-flow reactors, slurry reactors, ebullated bed reactors, jet flow reactors, mechanically agitated reactors, bubble columns, such as slurry bubble columns and external recycle loop reactors. The use of these reactor vessels allows dilution of the reaction mixture to an extent that provides high degrees of selectivity to the desired glycol product (mainly ethylene and propylene glycols). In one embodiment, there is a single reactor vessel, which is preferably a CSTR.

There may be more than one reactor vessel used to carry out the process of the present invention. The more than one reactor vessels may be arranged in series, or may be arranged in parallel with respect to each other, or in any combination of parallel and series. In a further embodiment, two reactor vessels arranged in series, preferably the first reactor vessel is a CSTR, the output of which is supplied to a second reactor vessel, which is a plug-flow reactor. The advantage provided by such two reactor vessel embodiment is that the retro-aldol fragments produced in the CSTR have a further opportunity to undergo hydrogenation in the second reactor vessel, thereby increasing the glycol yield of the process. The second reactor vessel, which is a plug-flow reactor, is suitably a fixed-bed type reactor.

Preferably, the process of the present reaction takes place in the absence of air or oxygen. In order to achieve this, it is preferable that the atmosphere in the reactor vessel is evacuated after loading of any initial reactor vessel contents and before the reaction starts, and initially replaced with nitrogen gas. There may be more than one such nitrogen replacement step before the nitrogen gas is removed from the reactor vessel and replaced with hydrogen gas.

The process of the present invention takes place in the presence of hydrogen. To start the process, the reactor vessel is heated to a reaction temperature and further hydrogen gas is supplied to it under pressure. In the embodiment where there is a single reactor vessel, hydrogen gas is supplied into the reactor vessel at a pressure of at least 1 MPa, preferably at least 2 MPa, more preferably at least 3 MPa. Hydrogen gas is supplied into the reactor vessel at a pressure of at most 13 MPa, preferably at most 10 MPa, more preferably at most 8 MPa. In the embodiment where there are two reactor vessels arranged in series, hydrogen is supplied in to the CSTR at the same pressure range as for the single reactor (see above), and optionally hydrogen may also be supplied into the plug-flow reactor. If hydrogen is supplied into the plug-flow reactor, it is supplied at the same pressure range as for the single reactor (see above).

The process of the present invention takes place in the presence of hydrogen. The hydrogen gas is supplied to the reactor vessel at a pressure described above, and in a manner common in the art. In the embodiment with a single CSTR, preferably the hydrogen is bubbled through the reaction mixture in the CSTR. In the embodiment with a CSTR followed by a plug-flow reactor arranged in series, the hydrogen is bubbled through the reaction mixture in the CSTR, and in the plug-flow reactor, hydrogen is supplied into the reactor either in a counter-current or a co-current manner in relation the reaction mixture flow. In the embodiment with a CSTR followed by a plug-flow reactor arranged in series, optionally, the hydrogen is supplied via the hydrogen content of the material flowing out of the CSTR into the plug-flow reactor.

Irrespective of whether there is a single reactor vessel or there are two reactor vessels, the catalyst component with retro-aldol catalytic capabilities is supplied preferably into the CSTR. The weight ratio of the catalyst component with retro-aldol catalytic capabilities (based on the amount of metal in said composition) to the saccharide-containing feedstock is suitably in the range of from 1:100 to 1:1000.

Irrespective of whether there is a single reactor vessel or there are two reactor vessels, the catalyst precursor is supplied to each reactor vessel (in units of g metal per L reactor volume in each case) preferably at least at 0.01, more preferably at least at 0.1, even more preferably at least at 1 and most preferably at least 8. In such embodiment, the catalyst precursor is supplied to each reactor vessel (in units of g metal per L reactor volume in each case) preferably at most at 20, more preferably at most at 15, even more preferably at most at 12 and most preferably at most at 10.

In one embodiment, the catalyst precursor comprises ruthenium, which is supplied to each reactor vessel (in units of g metal per L reactor volume in each case) preferably at least at 0.01, more preferably at least at 0.1, even more preferably at least at 0.5. In such embodiment, the catalyst precursor comprising ruthenium is supplied to each reactor vessel (in units of g metal per L reactor volume in each case) preferably at most at 10, more preferably at most at 5, even more preferably at most at 2.

In another embodiment, the catalyst precursor comprises nickel, which is supplied to each reactor vessel (in units of g metal per L reactor volume in each case) preferably at least at 0.1, more preferably at least at 1, even more preferably at least at 5. In such embodiment, the catalyst precursor comprising nickel is supplied to each reactor vessel (in units of g metal per L reactor volume in each case) preferably at most at 20, more preferably at most at 15, even more preferably at most at 10.

In the embodiment where there is a single reactor vessel, the reaction temperature in the reactor vessel is suitably at least 130° C., preferably at least 150° C., more preferably at least 170° C., most preferably at least 190° C. In such embodiment, the temperature in the reactor vessel is suitably at most 300° C., preferably at most 280° C., more preferably at most 250° C., even more preferably at most 230° C. Preferably, the reactor vessel is heated to a temperature within these limits before addition of any reaction mixture and is controlled at such a temperature to facilitate the completion of the reaction.

In the embodiment with a CSTR followed by a plug-flow reactor arranged in series, the reaction temperature in the CSTR is suitably at least 130° C., preferably at least 150° C., more preferably at least 170° C., most preferably at least 190° C. The temperature in the reactor vessel is suitably at most 300° C., preferably at most 280° C., more preferably at most 250° C., even more preferably at most 230° C. In the embodiment with a CSTR followed by a plug-flow reactor arranged in series, the reaction temperature in the plug-flow reactor is suitably at least 50° C., preferably at least 60° C., more preferably at least 80° C., most preferably at least 90° C. The temperature in such reactor vessel is suitably at most 250° C., preferably at most 180° C., more preferably at most 150° C., even more preferably at most 120° C. Preferably, each reactor vessel is heated to a temperature within these limits before addition of any reaction mixture and is controlled at such a temperature to facilitate the completion of the reaction.

The pressure in the reactor vessel (if there is only one reactor vessel), or the reactor vessels (if there are more than one reactor vessel), in which the reaction mixture is contacted with hydrogen in the presence of the unsupported hydrogenation catalyst composition described herein is suitably at least 3 MPa, preferably at least 5 MPa, more preferably at least 7 MPa. The pressure in the reactor vessel, or the reactor vessels, is suitably at most 12 MPa, preferably at most 10 MPa, more preferably at most 8 MPa. Preferably, the reactor vessel is pressurised to a pressure within these limits by addition of hydrogen before addition of any reaction mixture and is maintained at such a pressure until all reaction is complete through on-going addition of hydrogen. In the embodiment where there are two reactor vessels arranged in series, a pressure differential in the range of from 0.1 MPa to 0.5 MPa exists across the plug-flow reactor to assist the flow of the liquid phase through the plug-flow reactor.

Irrespective of whether there is a single reactor vessel or there are two reactor vessels, in the process of the present invention the residence time of the reaction mixture in each reactor vessel is suitably at least 1 minute, preferably at least 2 minutes, more preferably at least 5 minutes. Suitably, the residence time of the reaction mixture in each reactor vessel is no more than 5 hours, preferably no more than 2 hours, more preferably no more than 1 hour.

In the embodiment where the catalyst component with retro-aldol catalytic capabilities comprises tungsten supported on a solid support (or a or a combination of solid supports), a problem observed by the inventors of the present application is that the association between tungsten and the solid support is insufficient, leading to leaching of the tungsten from the solid support, and mixing with the other components within the reactor vessel. In the embodiment where the catalyst component with retro-aldol catalytic capabilities comprises unsupported tungsten, by the nature of its operation as a homogeneous catalyst, tungsten is in a mixture with the other components within the reactor vessel. In both of these embodiments, the mixture of the tungsten compounds and complexes with the other components within the reactor vessel leads to the formation of insoluble compounds of tungsten, in particular insoluble oxides of tungsten. In particular, the mixture of the tungsten compounds and complexes with saccharide- and glycol-containing aqueous mixtures forms insoluble compounds of tungsten. Such insoluble compounds of tungsten are observed to stick to the pores of solid supports such as silica, alumina, zirconia, activated carbon or zeolites, as well as to the surface of other nano- and micro-entities with rough surface topologies. Where the insoluble compounds of tungsten stick to such pores or surfaces of catalytic entities, they irreversibly reduce the catalytic activity of the catalytic entities by preventing access of the reactants to the surface of the catalytic entity.

The inventors of the present invention believe that the physical form of the unsupported hydrogenation catalyst generated in the process of the present invention is micron-sized particles. This belief is based on the retention of a substantial amount of the unsupported hydrogenation catalyst by a 0.45 micron filter, when the reactor vessel content (taken during glycol production) is filtered through it. Although retained by such pore-sized filter, no significant sedimentation of the unsupported hydrogenation catalyst is observed if the reactor vessel content remains at 1×G, suggesting that the diameter of such particles is between 0.45 μm to approximately upper limit of about 10 μm. The approximate upper limit of about 10 μm is based on the assumption that above this diameter, in general particles are no longer able to participate in Brownian motion, and sediment.

The inventors further believe that the surface topology of the micron-sized particles is smooth and do not contain any significant pores, making them resistant to the attachment of insoluble compounds of tungsten on their surface. This allows the unsupported hydrogenation catalyst to be used in the same reactor vessel as the catalyst component with retro-aldol catalytic capabilities without the loss of any hydrogenation catalytic activity from such interaction.

The inventors of the processes of the present inventions have found that the resistance of the unsupported hydrogenation catalyst described herein to deactivation by the insoluble chemical species generated by the catalyst component with retro-aldol catalytic capabilities (whether supported or unsupported) provides a solution to the problem of the hydrogenation catalyst deactivation when glycols are prepared from a saccharide-containing feedstock in a single reaction vessel.

A further advantages of the unsupported hydrogenation catalyst prepared as described herein is that it functions with the advantages of both a homogeneous-type and a heterogeneous-type catalyst, but without their respective disadvantages. In particular the unsupported hydrogenation catalyst can be supplied to the reactor vessel with, and at the same time as, the reaction mixture. This overcomes the need to have any further means for catalyst introduction into the reactor vessel, simplifying the reactor setup. Further, it is retained in the reactor vessel by a simple filtration step, also negating the need to use complicated and expensive reactor setups. Therefore otherwise cumbersome solids handling and recovery of deactivated hydrogenation catalyst is solved, and reactor vessels designed for handling homogeneous liquids can be used, and the process of hydrogenation catalyst preparation is significantly simplified.

The present invention is further illustrated in the following Examples.

### EXAMPLES

Overview of the examples: In Example 1, the catalyst precursor was converted to the unsupported hydrogenation catalyst in the presence of hydrogen in a reactor vessel and its activity was assessed in the presence of a catalyst component with retro-aldol catalytic capabilities (sodium phosphotungstate), but in the absence of the saccharide-containing feedstock (glucose). In Example 2, activity of the unsupported hydrogenation catalyst was assessed in the presence of saccharide feedstock (glucose) and a catalyst component with retro-aldol catalytic capabilities. In Example 3, when further saccharide-containing feedstock (glucose) was added to the reactor vessel, more glycol product (e.g. MEG) was produced. In Example 4, a sample was taken from Example 1 reactor vessel content and filtered through a 0.45 μm pore-sized filter, and when mixed with saccharide-containing feedstock and the catalyst component with retro-aldol catalytic capabilities, it was observed that the level of glycol products (e.g. MEG) had diminished.

### Example 1: Formation of Unsupported Hydrogenation Catalyst and its Background Activity

A 60 ml Hastelloy C22 autoclave (Medimex), equipped with a hollow-shaft gas stirrer, was loaded with 15 g water and 15 g glycerin, 60.1 mg sodium phosphotungstate (Aldrich) and 7.0 mg ruthenium(III)acetylacetonate (catalyst precursor; Merck), pre-dissolved in a water/glycerin mixture (Table 1). The reactor vessel was pressurized with nitrogen to 5 barg and depressurized to atmospheric for 3 times to remove oxygen, then pressurized with hydrogen to 40 barg at room temperature. The temperature was increased to 195° C., the total pressure raised with hydrogen to 80 barg and a stirring rate of 1450 rpm was applied. After 60 minutes the reactor vessel was allowed to cool down to room temperature, opened and a sample taken for analysis (Table 2). Glycerin appeared to be stable, as only traces of products are formed, indicating that glycerin can be applied as an inert solvent. Any glycols formed in the subsequent examples do not originate from glycerin under the concentrations and conditions applied.

### Example 2: Activity of the Unsupported Hydrogenation Catalyst from Example 1 in the Presence of Both a Saccharide Feedstock and a Catalyst Component with Retro-Aldol Catalytic Capabilities

A 60 ml Hastelloy C22 autoclave (Medimex), equipped with a hollow-shaft gas stirrer, was loaded with 14.2 g reactor vessel effluent of Example 1. Water and glycerin were added in equal weight amounts to a total of 15.2 g reactor vessel content, as well as 0.3 g of glucose (Millipore). The reactor vessel was pressurized with nitrogen to 5 barg and depressurized to atmospheric for 3 times to remove oxygen, then pressurized with hydrogen to 40 barg at room temperature. The temperature was increased to 195° C., the total pressure raised to 80 barg and a stirring rate of 1450 rpm was applied. After 60 minutes the reactor vessel was allowed to cool down to room temperature, opened and a sample taken for analysis (Table 2). This example demonstrates catalytic activity of the liquor obtained from Example 1 for the conversion of glucose to glycols.

### Example 3: Second Run with Further Glucose Added

The reactor vessel content of Example 2 was obtained and 0.3 g of glucose (Millipore) was added. Some water and glycerin were added in equal weight amounts to obtain a total of 30.2 g reactor vessel content. The reactor vessel was pressurized with nitrogen to 5 barg and depressurized to atmospheric for 3 times to remove oxygen, then pressurized with hydrogen to 40 barg at room temperature. The temperature was increased to 195° C., the total pressure raised with hydrogen to 80 barg and a stirring rate of 1450 rpm was applied. After 90 minutes the reactor vessel was allowed to cool down to room temperature, opened and a sample taken for analysis (Table 2). This example demonstrates catalytic activity of the liquor obtained from Example 2 for the conversion of glucose to glycols. The liquid was filtered through a 0.45 micron filter and the ruthenium content was measured to be 1.4 ppmw Ru, as measured by Inductive Coupled Plasma analysis. The original Ru intake corresponds to 21.5 ppm Ru, indicating that the majority of the original Ru(acac)3 intake is precipitated as particles larger than 0.45 micron.

### Example 4: 50% Reactor Vessel Effluent Obtained from Example 1a, Now Filtered Through a 0.45 Micron Filter

A 60 ml Hastelloy C22 autoclave (Medimex), equipped with a hollow-shaft gas stirrer, was loaded with 11.3 g reactor vessel effluent of Example 1, filtered through a 0.45 micron filter and 0.3 g glucose (Millipore). Water/glycerin 1:1 was added to a total of 30.3 g reactor vessel content (Table 1). The reactor vessel was pressurized with nitrogen to 5 barg and depressurized to atmospheric for 3 times to remove oxygen, then pressurized with hydrogen to 40 barg at room temperature. The temperature was increased to 195° C., the total pressure raised with hydrogen to 80 barg and a stirring rate of 1450 rpm was applied. After 90 min the reactor vessel was allowed to cool down to room temperature, opened and a sample taken for analysis (Table 2). The filtration step resulted in a significant reduction of hydrogenation catalytic activity, as indicated by the presence of hydroxyacetone and 1-hydroxy-2-butanone (Table 2), suggesting that the hydrogenation catalytic activity is associated with particles that can be retained by the 0.45 micron filter. Nevertheless, some MEG was observed to be produced, and the inventor of the present process believe that such MEG was not produced from the filtrate, but from the unsupported hydrogenation catalyst which remained associated with the reactor vessel walls following the single flush with 30 g demi water.

### LEGEND

MEG: 1,2-ethylene glycol

MPG: 1,2-propylene glycol

HA: hydroxyacetone

1,2-BDO: 1,2-dihydroxybutane

1H2BO: 1-hydroxy-2-butanone

% (w/w): weight percent, basis glycerin (Example 1) or glucose (all other examples), defined by product weight/glycerin weight*100% or product weight/glucose weight*100%.

TABLE 1
Feed Input

| | | water: | | | | | | |
|---|---|---|---|---|---|---|---|---|
| | | glycerin | | | | | | |
| | glucose | 1:1 | | | | | total | glucose |
| | intake | intake | effluent | effluent | | | intake | conc. |
| Example | (g) | (g) | intake | treatment | W (mg) | Ru (mg) | (g) | (w/w %) |
| 1 | 0 | 30.10 | | | 60.1 | 7.0 | 30.16 | |
| 2 | 0.3 | 0.8 | 14.2 g of | not | 28.4 | 3.3 | 15.20 | 1.96 |
| | | | Example 1 | filtered | | | | |
| 3 | 0.3 | 18.5 | 11.5 g of | not | 22.0 | 2.6 | 30.20 | 0.99 |
| | | | Example 2 | filtered | | | | |
| 4 | 0.3 | 18.8 | 11.3 g of | filtered | 21.9 | 2.7 | 30.30 | 0.99 |
| | | | Example 1 | | | | | |

TABLE 2
Product Yields

| | MEG | MPG | HA | 1,2BDO | 1H2BO | Total |
|---|---|---|---|---|---|---|
| Example | % (w/w) | % (w/w) | % (w/w) | % (w/w) | % (w/w) | % (w/w) |
| 1 | 0.01 | 0.06 | 0.03 | 0.00 | 0.00 | 0.10 |
| 2 | 23.02 | 3.68 | 5.06 | 1.47 | 7.93 | 41.15 |
| 3 | 30.08 | 5.05 | 5.42 | 2.40 | 6.19 | 49.14 |
| 4 | 4.96 | 1.66 | 5.75 | 0.23 | 5.45 | 18.06 |

1. A process for the production of glycols comprising the step of adding to a reactor vessel a saccharide-containing feedstock, a solvent, hydrogen, a retro-aldol catalyst composition and a catalyst precursor and maintaining the reactor vessel at a temperature and a pressure, wherein the catalyst precursor comprises one or more cations selected from groups 8, 9, 10 and 11 of the periodic table, and wherein the catalyst precursor is reduced in the presence of hydrogen in the reactor vessel into an unsupported hydrogenation catalyst.

2. The process claimed in claim 1, wherein the glycols comprise ethylene glycol and 1, 2-propylene glycol.

3. The process claimed in claim 1, wherein the saccharide-containing feedstock comprises one or more saccharide selected from the group consisting of monosaccharides, disaccharides, oligosaccharides and polysaccharides.

4. The process claimed in claim 1, wherein the solvent is water, or a C1, C2, C3, C4, C5 or a C6 alcohol or polyalcohol, or any combination of mixtures thereof.

5. The process claimed in claim 1, wherein the cation is selected from a group consisting of iron, ruthenium, cobalt, rhodium, nickel, palladium and platinum.

6. The process claimed in claim 1, wherein the cation is selected from a group consisting of ruthenium, nickel, palladium and platinum.

7. The process claimed in claim 1, wherein the catalyst precursor comprises ruthenium cations.

8. The process claimed in claim 1, wherein the catalyst precursor comprises an anion selected from a group consisting of carboxylates, acetylacetonate and inorganic anions, which in all cases forms a salt or a complex that is soluble in a solvent mixture comprising the saccharide-containing feedstock, the solvent and the glycols.

9. The process according to claim 1, wherein the catalyst precursor comprises acetylacetonate.

10. The process according to claim 1, wherein the retro-aldol catalyst composition comprises tungsten.
